(19) United States
(12) Patent Application Publication
(10) Pub. No.: US 2009/0156888 A1
(43) Pub. Date: Jun. 18, 2009

(54) Method and apparatus for preventing, controlling and/or treating eye fatigue and myopia

(75) Inventors: Xianhui Su, Lombard, IL (US); Shigong Su, Lombard, IL (US); Xianhui Su, Lombard, IL (US)

Correspondence Address: Xianhui Su, 106 Oakton Dr, Lombard, IL, 60148, US

(73) Assignee: Xianhui Su, Lombard, IL (US)

(21) Appl. No.: 12/157,071

(22) Filed: Jun. 7, 2008

Related U.S. Application Data
(60) Provisional application No. 60/997,709, filed on Oct. 4, 2007.

(51) Int. Cl. A61M 21/00

(52) U.S. Cl. 600/27

(57) ABSTRACT

An apparatus and method for preventing, controlling and/or treating vision disorders, such as eye fatigue and myopia, comprises a panel or a plurality of panels with a contrast stimulus or contrast stimuli presented to a peripheral vision or peripheral visions. A contrast stimulus may be a spatial frequency contrast, a luminance contrast, a translucency/transparency contrast, a depth contrast or a spectral contrast. The panel is configured to fit on screens, monitors, televisions, displays and the like. The panel may be adapted to fit on hats, glasses, the head, and the like. The panel may be constructed to be portable and stand alone, to be used while viewing a primary object, such as a visual display monitor, a book, or the like.

### TECHNICAL FIELD

This invention relates to a method and an apparatus for preventing, controlling and/or treating vision disorders, irregularities and abnormalities.

### BACKGROUND ART

With the advent of computers and the explosion of the internet, more time is spent staring into computer screens, monitors, and other visual devices. Whether its conducting business, communicating with friends, or playing video games, people of all ages have succumbed to staring into some sort of visual display monitor for many hours for the sake of efficiency, information and entertainment. These benefits, however, do not come without a cost. Prolonged hours of staring into a visual display monitor results in disorders, irregularities, and abnormalities in vision, such as eye fatigue and myopia or nearsightedness. These disorders, irregularities, and abnormalities are also common from prolonged focus on other materials such as books, notes, papers, and the like.

Current treatment for these types of vision disorders includes corrective lenses and surgery. Surgical procedures although greatly improved are still expensive and are subject to a variety of risks and complications. Corrective lenses, whether glasses or contact lenses, although cheaper than surgery are still quite costly due to replacement, maintenance and upgrades. In addition, corrective lenses are inconvenient. Glasses can be cumbersome or uncomfortable on the face. Contact lenses often times dry out causing great discomfort.

Therefore, there exists a need for preventing and/or treating eye fatigue and myopia that is affordable, easy to maintain, and convenient.

Please refer to appendix on background information regarding converting 3D orthogonal (x, y, z) coordinates to spherical (q, e, d) coordinates.

### SUMMARY OF INVENTION

A method and apparatus for preventing, controlling and/or treating accommodation lag, accommodation fatigue, eye fatigue, and myopia comprises a visual stimulus or a plurality of visual stimuli with contrast pattern(s) placed in the peripheral visual field(s). The visual stimulus (stimuli) can be any pattern, design, character, or picture with a contrast(s). The contrast patterns can be a spectral contrast, a luminance contrast, a translucency/transparency contrast, a depth contrast, or any combination thereof. The visual stimuli may be displayed on a panel or projected as a light form. The visual stimulus may be placed on the visual display monitor, worn by the user, or may be a portable, free standing apparatus. The positioning of the visual stimuli relative to the visual display monitor and/or the user may be adjustable.

### BRIEF DESCRIPTION OF DRAWINGS

FIG. 1A is a top perspective view of an embodiment of the current invention;

FIG. 1B is a side perspective view of an embodiment of the current invention;

FIG. 2A-2D are embodiments of visual stimuli;

FIG. 2E is other examples of visual stimuli;

FIG. 3A is a front view of an embodiment of the current invention fully expanded;

FIG. 3B is a front view of the embodiment in FIG. 3A in a collapsed state;

FIG. 3C is a front view of an embodiment of the current invention;

FIG. 3D is a front view of the embodiment in FIG. 3C fully expanded;

FIG. 3E is a front view of the embodiment in FIG. 3C collapsed and folded up for traveling;

FIG. 3Fa is another possible frontal view of the embodiment in FIG. 3C.

FIG. 3Fb is the frontal view of the embodiment in FIG. 3Fa after adjustments.

FIG. 3G is the frontal view of the embodiment in FIG. 3Fa using first support member 304 and a second support member 305.

FIG. 3H is the frontal view of the first support member 304, the second support member 305 and the third support member 307.

FIG. 3I is the frontal view after assemble first support member 304, the second support member 305 and the third support member 307 together.

FIG. 3J is the top view of the first support member 304, the second support member 305 and the third support member 307.

FIG. 3K is the top view after assemble first support member 304, the second support member 305 and the third support member 307 together.

FIG. 3L is the right side view after assemble first support member 304, the second support member 305 and the third support member 307 together.

FIG. 4A is another embodiment of the current invention;

FIG. 4B is the embodiment shown in FIG. 4A attached to a wider screen by adjusting the alignment of the superior panels;

FIG. 4C is the embodiment shown in FIG. 4B with smaller visual stimulus angles;

FIG. 4D is the embodiment shown in FIG. 4B with larger visual stimulus angles;

FIG. 4E is a side view of the embodiment shown in FIG. 4B;

FIG. 4F is a side view of the embodiment shown in FIG. 4C;

FIG. 4G is a side view of the embodiment shown in FIG. 4D;

FIG. 4H is an embodiment that has the same panels as the embodiment shown in FIG. 4B, but the panels are positioned differently, wherein the left superior panel and the right lateral panel swapped positions, and the left lateral panel and the right superior panel swapped positions.

FIG. 4I is a side view of the embodiment shown in FIG. 4H;

FIG. 5A is a top view of an embodiment of the current invention;

FIG. 5B is a top view of the embodiment in FIG. 5A assembled;

FIG. 5C is a side view of an embodiment of the current invention;

FIG. 5D is a front view of the embodiment in FIG. 5C;

FIG. 6 is a top view of an embodiment of the current invention;

FIG. 7A is a front view of another embodiment of the current invention;

FIG. 7B is a side view of another embodiment of the current invention.

FIG. 8A is a top view of an embodiment of the current invention;

FIG. 8B is a front view of the embodiment in FIG. 8A;

FIG. 8C is a side view of the embodiment in FIG. 8A;

FIG. 8D is a top view of the embodiment in FIG. 8A that is adjusted to have smaller visual stimuli angles;

FIG. 8E is a front view of the embodiment in FIG. 8D;

FIG. 8F is a side view of the embodiment in FIG. 8D;

FIG. 8G is the partially folded plurality of panels used in the embodiment in FIG. 8A;

FIG. 8H is the folded plurality of panels used in the embodiment in FIG. 8A;

FIG. 9 is the drawing of the 3-D (x, y, z) orthogonal coordinates, and the spherical (q, e, d) coordinates.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the invention.

Vision disorders, irregularities, and abnormalities include, without limitation, myopia or near-sightedness, accommodation fatigue, accommodation lag, accommodation error, intra/extraocular muscle fatigue and abnormalities, and amblyopia. Such vision disorders make it difficult for a viewer to focus on a primary object. Thus, the devices and methods described herein are designed to treat, control and/or prevent these disorders by helping the eyes to focus (accommodate) on the primary object.

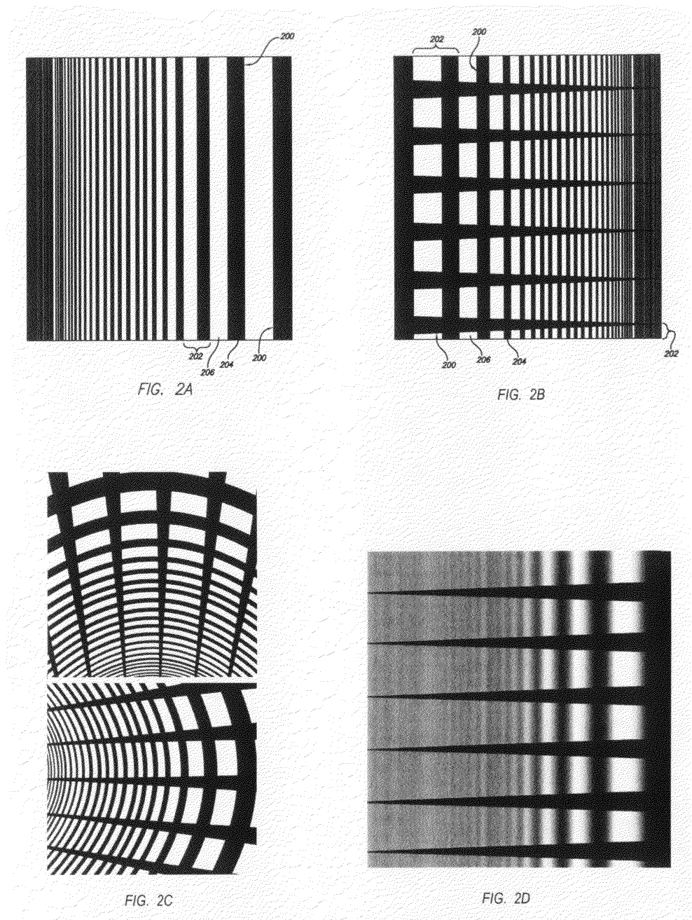

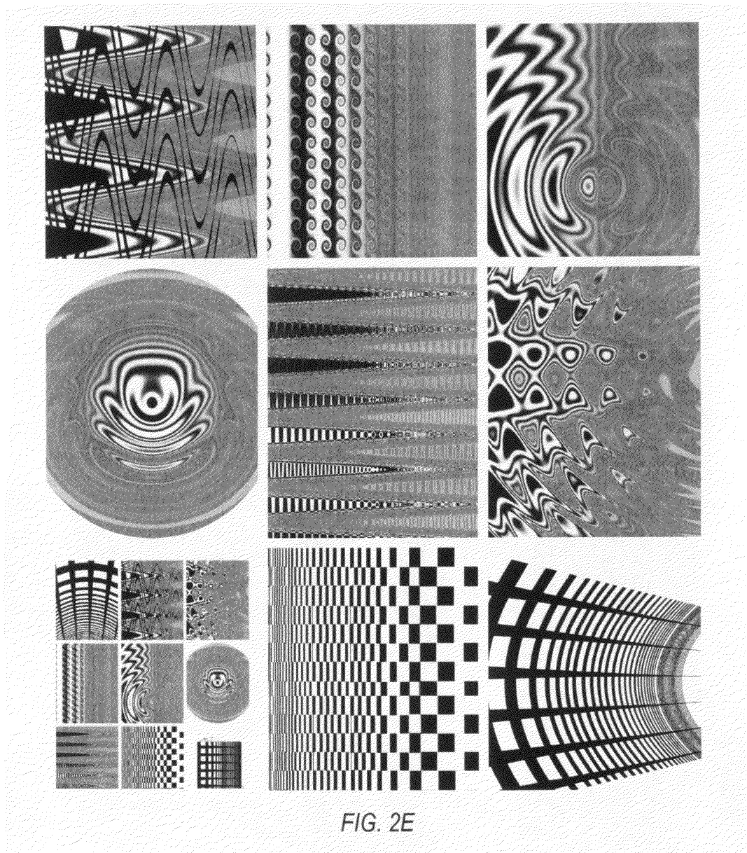

This invention is directed towards a method and apparatus for treating vision disorders by providing a visual stimulus or visual stimuli 102 to a peripheral visual field or peripheral visual fields 104, wherein the visual stimulus or visual stimuli comprises a contrast pattern or contrast patterns 200. Examples of possible contrast patterns are show in FIG. 2A through FIG. 2E. A contrast is the noticeable difference between two areas or portions with dissimilar features. The dissimilar feature or characteristic can be a difference in color, luminance, translucency/transparency, or distance from the eye, and the like, or any combination thereof. The transition from a first area 204 to a second area 206 can be gradual or abrupt.

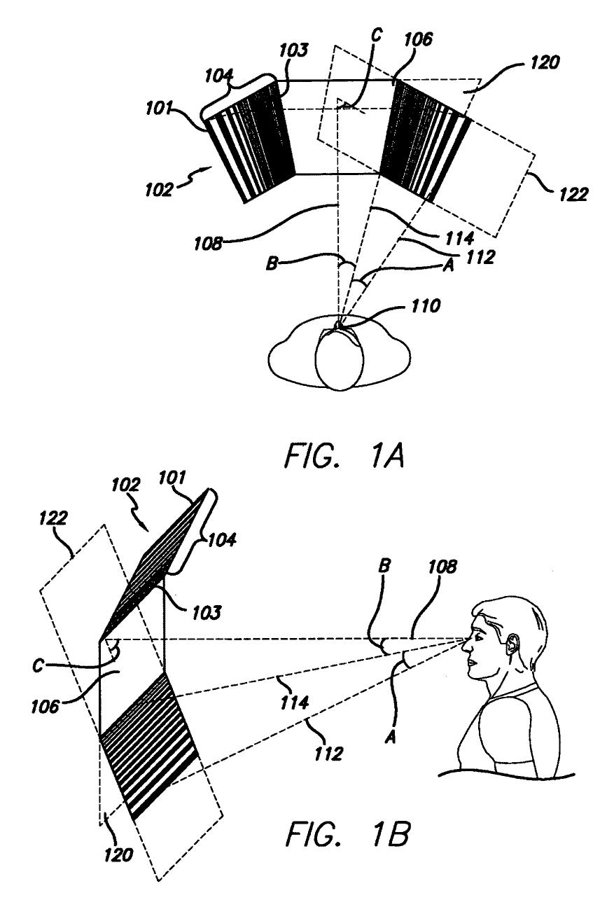

The peripheral visual field 104 is defined as the field of view from the field adjacent to a primary object 106 to up to 150° of peripheral eccentricity e in the spherical (q, e, d) coordinate system defined in the appendix. Thus, the peripheral visual field and peripheral visual fields 104 may be above, below, next to, or around the primary object 106, or any combination thereof, as shown in FIGS. 1A and 1B. A viewer focusing on the primary object 106 defines a center of gaze or a central visual axis 108, which is an imaginary line from the general area the viewer is visually focused on to a point midway between the eyes, referred to as the central visual point 110. The visual stimulus 102 comprises a distal end 101, a proximal end 103, and a contrast pattern(s) 200. The proximal end 103 is the side of the visual stimulus 102 closer to the primary object 106. The distal end 101 is the side farther from the primary object 106. The distal peripheral visual angle A is the peripheral eccentricity angle created by the central visual axis 108 and ray 112 projecting from the central visual point 110 towards a point on the distal end 101 of the visual stimulus 102 with the central visual point 110 as the vertex. (Note: different points on the distal end 101 of the visual stimulus 102 may have different distal peripheral visual angles) Thus, the distal peripheral visual angle A is a function of the size of the visual stimulus 102 and the distance the visual stimulus 102 away from the eye. For example, two visual stimuli of different sizes will have different distal peripheral visual angles at the same distance from the eye. As another example, a single visual stimulus 102 placed near the eye will have a different distal peripheral visual angle A than when placed farther away from the eye even though the size of the visual stimulus 102 has not changed. As shown in FIGS. 1A and 1B, a proximal peripheral visual angle B is the peripheral eccentricity angle created by the central visual axis 108 and a ray 114 directed towards a point on the proximal end 103 of the visual stimulus 102, with the central visual point 110 as the vertex. (Note: different points on the proximal end 103 of a visual stimulus 102 may have different proximal peripheral visual angles.

In some embodiments, the contrast pattern(s) 200 may be created by a spectral or color contrast. In other embodiments, the contrast pattern(s) 200 are created by a luminance contrast. In other embodiments, the contrast patterns 200 are created by a combination of spectral contrast and luminance contrast, for example, a natural photograph or picture.

A spectral contrast is any contrast created by a difference in color. For example, a red area adjacent to a blue area creates a spectral contrast. Even a different shade or hue of the same color can create a spectral contrast. For example, light with a wavelength of 650 nanometers will be perceived as red to a viewer. Light with a wavelength of 700 nanometers will also be perceived as red to a viewer. When these two shades of red are placed next to each other, a contrast pattern 200 will be created.

Color as used herein refers to any light or reflection of light that can be perceived by the eye. In general, this includes, but is not limited to, the visible spectrum of light ranging from approximately 350 nanometers to approximately 750 nanometer wavelengths including any combination thereof. Color as used herein also includes black, white, all shades of grey in between, and/or any combination of wavelengths.

In some embodiments, the spectral contrast is created from two (or more) sets of colors differing in the spectral analysis of the color spectrum by at least 15 nanometers. In other words, the spectral contrast may be created by a first set of spectral frequencies at a first area and a second set of spectral frequencies at a second area, wherein the first and second sets of spectral frequencies each consists of a single spectral frequency, or a combination of spectral frequencies; and wherein at least one of the color spectral frequency from the first set is different from one of the color spectral frequency from the second set by at least approximately 15 nanometers. In another word, the difference between the first set and second set of color spectral frequencies is at least approximately 15 nanometers. Preferably, the difference between the first set and second set of color spectral frequencies is at least approximately 20 nanometers. More preferably, the difference between the first set and second set of color spectral frequencies is at least approximately 30 nanometers. More preferably, the difference between the first set and second set of color spectral frequencies is at least approximately 40 nanometers.

For the intended purpose of preventing, controlling and/or treating myopia, it is very important to provide good contrast pattern(s) as the visual stimulus or visual stimuli to prevent the empty field effect. When looking at an empty field, such as a white screen, or in a dark room, the len(s) of the eye(s) of a normal person will be at approximately 1+ diopter. This effect is called the empty field effect. When looking at an object at infinity, the lens of a normal human eye will be at approximately zero diopter, while looking at an object at 20 cm away from the eye corresponds to approximately 5+ diopters.

The spatial frequency spectral analysis of a contrast pattern 200 can be characterized by a single or a range of spatial frequencies expressed as cycles per degree of peripheral eccentricity and/or cycles per degree axle deviation, or in cycles per degree in any direction. A cycle 202 constitutes the two adjacent areas or portions with dissimilar features defining the contrast pattern.

In some embodiments, the density of the contrast cycles 202 changes as a function of the distance from the contrast cycle 202 to the primary object 106. For example, the number of contrast cycles 202 in a given area at the distal end 101 of a visual stimulus 102 may be fewer than the number of contrast cycles 202 at the proximal end 103. Thus, panning from the distal end 101 of the visual stimulus towards the proximal end 103, the density of the contrast cycles 202 increases.

The density of contrast cycle 202 in a given area may be altered by increasing/decreasing the number of first and/or second uniform areas in a given area. This can be accomplished by decreasing/increasing the thickness of the first uniform area, decreasing/increasing the thickness of the second uniform area, or decreasing/increasing the thickness of both uniform areas.

Luminance contrast is any contrast created by a difference in brightness. For example, an area having a luminance of 50 lux compared to an area having a luminance of 100 lux creates a luminance contrast. A luminance contrast can be quantified using the equation:

Luminance contrast=(Maximum Luminance−Minimum Luminance)/(Maximum Luminance+Minimum Luminance).

The range of luminance contrasts that is relevant for preventing, controlling and/or treating myopia is between approximately 10% to approximately 100%. Preferably the range of luminance contrasts is between approximately 20% to approximately 100%. More preferably the range of luminance contrasts is between approximately 50% to approximately 100%.

In some embodiments, the luminance can be defined relatively to a white color, such as a white piece of paper at the same position and orientation.

A relative luminance at coordinate (q_{b}, e_{b} dᵢ) is defined by the equation:

Relative luminance(q_{b}e_{b}dᵢ)=luminance(q_{b}e_{b}dᵢ)/luminance of white color(q_{b}e_{b}dᵢ);

where luminance (q_{b} e_{b} dᵢ) is the luminance at coordinate (q_{b} e_{b} dᵢ), and luminance of white color (q_{b} e_{b} dᵢ) is the luminance of white color at coordinate (q_{b} e_{b} dᵢ).

A mean relative luminance is defined as the simple average of the relative luminance of a visual stimulus or visual stimuli;

The mean relative luminance may range from approximately 5% to approximately 99%. Preferably, the mean relative luminance ranges from approximately 10% to approximately 99%. More preferably, the mean relative luminance ranges from approximately 20% to approximately 95%. More preferably, the mean relative luminance ranges from approximately 25% to approximately 85%.

A standard deviation of relative luminance can be defined by the equation:

Standard deviation of relative luminance=statistical standard deviation(Relative luminance(q,e,d))

A standard deviation of relative luminance of the relevant spatial frequency of a contrast pattern can be defined by the equation:

Standard deviation of relative luminance of relevant spatial frequency=standard deviatior(spatial_frequency_band_pass_filter_{—}0.1_to_{—}10_degree_per_cycle(Relative luminance(q,e,d)))

The relevant spatial frequencies of a contrast pattern for preventing, controlling, and/or treating myopia are spatial frequencies between approximately 0.1 and approximately 10 cycles per degree in any direction. The relevant spatial frequencies of a contrast pattern or contrast patterns can be determined by applying a band pass spatial frequency filter on the said contrast pattern(s) which pass through spatial frequencies between approximately 0.1 to approximately 10 cycles per degree, and filter out spatial frequencies that are either less than approximately 0.1 cycle per degree or greater than approximately 10 cycles per degree. This band pass filter process can be performed by using Adobe Photoshop(c) software. The output of the said spatial frequency band pass filter (from approximately 0.1 to approximately 10 cycles per degree) is defined as the relevant spatial frequencies for a given contrast pattern or contrast patterns. Preferably, the standard deviation of the relative luminance of the relevant spatial frequencies of a contrast pattern (or contrast patterns) is at least approximately 2.5%; More preferably, the standard deviation of the relative luminance of the relevant spatial frequencies of a contrast pattern (or contrast patterns) is at least approximately 5%; More preferably, the standard deviation of the relative luminance of the relevant spatial frequencies of a contrast pattern (or contrast patterns) is at least approximately 7.5%; More preferably, the standard deviation of the relative luminance of the relevant spatial frequencies of a contrast pattern (or contrast patterns) is at least approximately 10%; More preferably, the standard deviation of the relative luminance of the relevant spatial frequencies of a contrast pattern (or contrast patterns) is at least approximately 12.5%; More preferably, the standard deviation of the relative luminance of the relevant spatial frequencies of a contrast pattern (or contrast patterns) is at least approximately 15%; More preferably, the standard deviation of the relative luminance of the relevant spatial frequencies of a contrast pattern (or contrast patterns) is at least approximately 20%; More preferably, the standard deviation of the relative luminance of the relevant spatial frequencies of a contrast pattern (or contrast patterns) is at least approximately 25%.

A relative-luminance-contrast is defined by the equation:

relative-luminance-contrast=Standard deviation of relative luminance/mean relative luminance;

When the standard deviation of relative luminance is less than 5%, the relative-luminance-contrast is at least 10%; Preferably, when the standard deviation of relative luminance is less than 5%, the relative-luminance-contrast is at least 15%; More preferably, when the standard deviation of relative luminance is less than 5%, the relative-luminance-contrast is at least 20%; More preferably, when the standard deviation of relative luminance is less than 5%, the relative-luminance-contrast is at least 25%; More preferably, when the standard deviation of relative luminance is less than 5%, the relative-luminance-contrast is at least 30%; More preferably, when the standard deviation of relative luminance is less than 5%, the relative-luminance-contrast is at least 35%;

In addition, the contrast patterns 200 can be any shape, design, pattern or the like. For example, the contrast patterns may be created by any pattern (natural or man-made) with at least two different colors, grades of luminance, grades of translucencies/opaqueness, depths, or any combination thereof, wherein depth is defined as the distance from a point on a visual stimulus to the central visual point 110. The spatial frequency or frequencies of the visual stimulus can be uniform or non-uniform. The contrast patterns 200 may be created by letters, characters, numbers, dots, lines, grids, checkerboard, geometric shape, geometric patterns, photographs, and the like with regular or irregular sizes, placed in regular or irregular patterns or directions, or any combination thereof. In embodiments utilizing lines, the lines may be of straight, curved, varying thicknesses, parallel, non-parallel, tapering, criss-crossing, and so on.

Using the spherical q, e, d coordinate system (defined in the appendix), One type of pattern may be defined by, but is not limited to, a spatial frequency of X/(1+10*e) with unit of cycle per degree of peripheral eccentricity and/or cycle per degree of axle deviation, where X may be a function, a numbers or numbers ranging from approximately 0.5 to approximately 10, and e is the peripheral eccentricity as shown in appendix FIG. 1. Preferably, X ranges from approximately 1 to approximately 6. More preferably, X is equal to the number pi, π (3.1415926). X may be a constant or a variable. For example, X may change as a function of the peripheral eccentricity e. Peripheral eccentricity e is expressed in radians, or degree of peripheral eccentricity*PI/180.

In other embodiments, the contrast patterns 200 can result from photographs, pictures, drawings, paintings, lights, holograms, translucencies/opaqueness, or any combination thereof.

The distance from any part of a visual stimulus to the center of pupil of an eye is at least approximately 7 cm. Preferably, the ratio of distance from any part of a visual stimulus to the center of pupil of an eye vs. the distance between the primary object and the eye is less than or equal to approximately 1, and the distance from any part of a visual stimulus to the center of pupil of an eye is greater than or equal to approximately 7 cm. More preferably, the distance from any part of a visual stimulus to the center of pupil of an eye is greater than or equal to approximately 10 cm and less than the distance between the primary object and the eye.

The edges of the visual stimulus may be any shape or design and not necessarily a straight edge. The distance between the primary object and any point on the edge of a visual stimulus may vary along the edge. The distal peripheral visual angle A may vary along the edge. The proximal peripheral visual angle B may vary along the edge. There may be a lot of different values of proximal peripheral visual angle B or distal peripheral visual angle A in a single embodiment.

For the attachable visual stimuli embodiments, the proximal end maybe directly attached to the primary object 106. For other embodiments, such as wearable embodiments, portable embodiments, stand along embodiments, etc, the visual stimuli 102 may be presented to the user from a proximal peripheral visual angle B of as low as approximately zero degree to a distal peripheral visual angle A of up to approximately 150 degree. More specifically, the proximal. peripheral visual angles may range from approximately zero degree to approximately 80 degree. Preferably, the proximal peripheral visual angles range from approximately 5 degree or immediately adjacent to the primary object to approximately 70 degree. More preferably, the proximal peripheral visual angles may range from approximately 5 degree or immediately adjacent to the primary object to approximately 50 degree. More preferably, the proximal peripheral visual angles may range from approximately 5 degree or immediately adjacent to the primary object to approximately 30 degree. More preferably, the majority of the proximal peripheral visual angles are approximately 5 degree or immediately adjacent to the primary object. The distal peripheral visual angles may range from any angle greater than the proximal peripheral visual angles to approximately 150 degree. More preferably, the distal peripheral visual angles are any angle greater than 45 degree. More preferably, the distal peripheral visual angles are any angle greater than 60 degree. More preferably, the distal peripheral visual angles are any angle greater than 80 degree. More preferably, the distal peripheral visual angles are any angle greater than 90 degree.

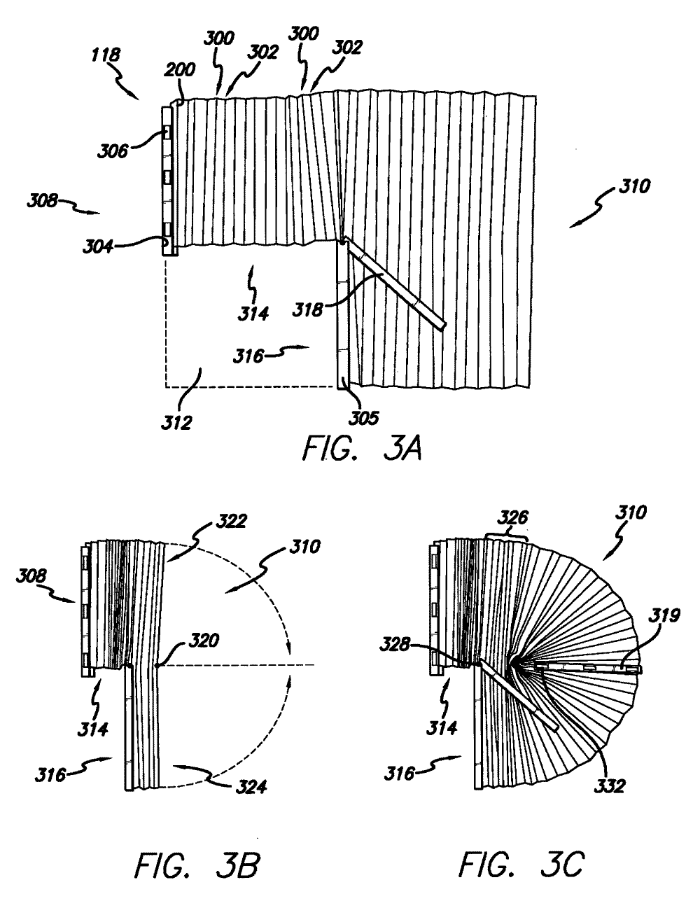

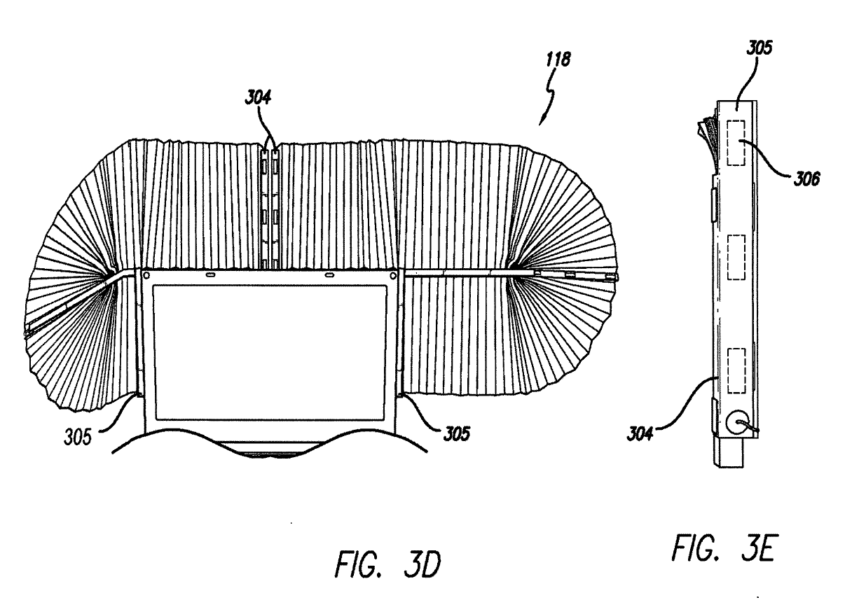

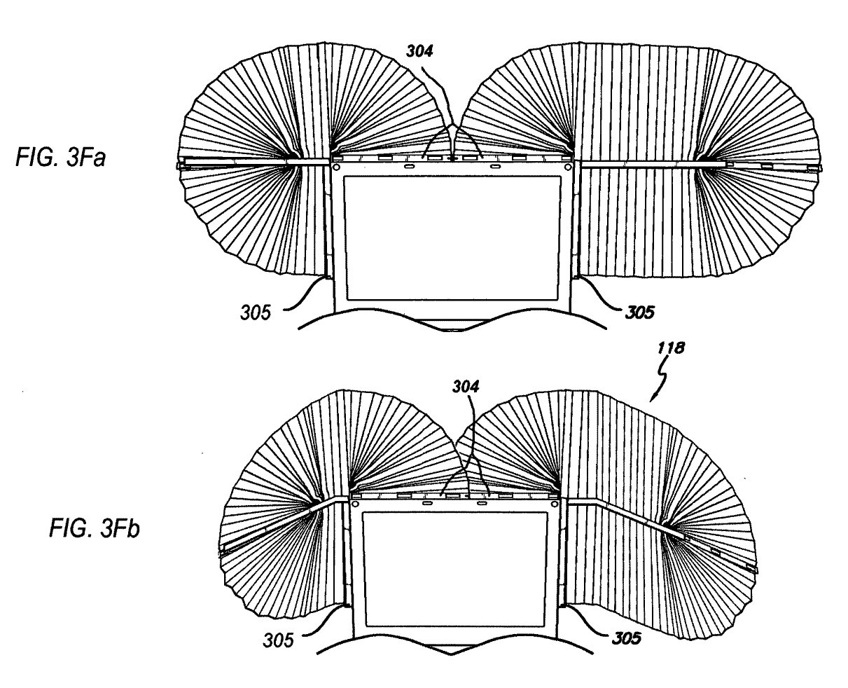

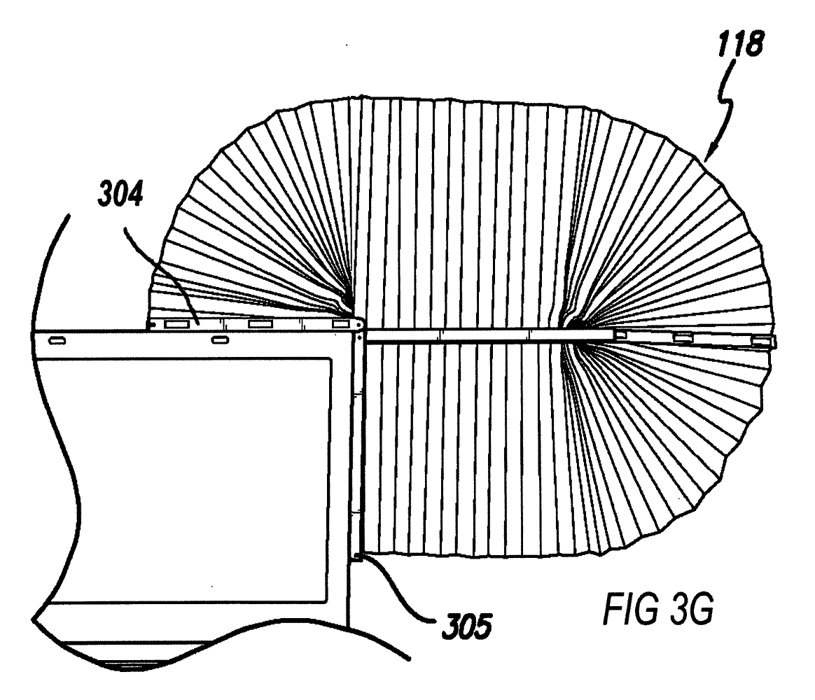

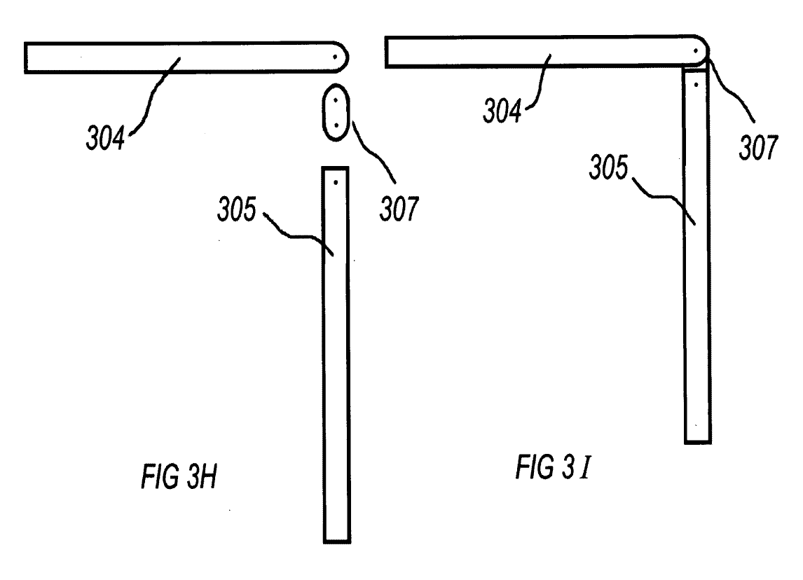

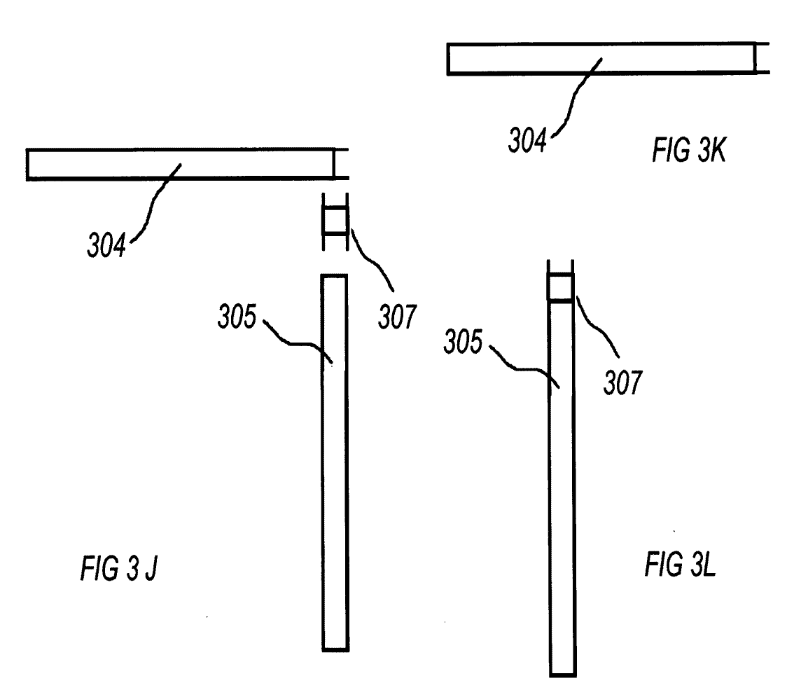

The visual stimulus angle C is defined as the angle from the central visual axis 108 to a tangential plane 122 defined by a section of the visual stimulus 102. Different sections (areas) of the visual stimuli 102 may have different visual stimulus angles. The visual stimulus angles may be ranges from approximately −90 degree to approximately 270 degree. The visual stimulus angle C of a section may be approximately zero degree, where the contrast presenting surface (visible side) of the said section of the visual stimulus faces toward the central visual axis 108 and the plane of the visual stimulus 122 is parallel to the central visual axis 108. The visual stimulus angle C of a section may be approximately 90 degree, where the plane of the visual stimulus 122 is perpendicular to the central visual axis 108. In some embodiments, such as collapsible embodiment shown in FIGS. 3A-3E, the visual stimulus angle C of a section may be greater than 90 degree, where the contrast presenting surface (visible side) of the said section of the visual stimulus faces away from the central visual axis 108. In some embodiments, the visual stimulus angle C of a section may be less than zero degree, where the contrast presenting surface of the said section of the visual stimulus faces toward the primary object 106.

The average visual stimulus angle of the portion of a visual stimulus, which is located between the proximal peripheral visual angles and 80 degree of peripheral eccentricity, is the average of the visual stimulus angles of visual stimulus 102 located between the proximal peripheral visual angles and 80 degree of peripheral eccentricity. It can be calculated by using the following formula:

C=visual stimulus angle C

TAVSB80E(C)=total area of the visual stimulus located between the proximal peripheral visual angles and 80 degree of peripheral eccentricity that have approximately visual stimulus angle C

TA80=total area of the visual stimulus located between the proximal peripheral visual angles and 80 degree of peripheral eccentricity

Average visual stimulus angle between the proximal peripheral visual angles and 80 degree of peripheral eccentricity can be calculated as

$$\frac{{\sum }_{C = - 90}^{270} C * TAVSB 80 E ( C )}{TA 80}$$

For example, the total area of a visual stimulus between the proximal peripheral visual angles and 80 degree of peripheral eccentricity is 2000 inch²; 500 inch² of the visual stimulus between the proximal peripheral visual angles and 80 degree of peripheral eccentricity has a visual stimulus angle C of approximately 30 degree, and 500 inch² of the visual stimulus between the proximal peripheral visual angles and 80 degree of peripheral eccentricity has a visual stimulus angle C of approximately 17.5 degree and the remaining 1000 inch² of the visual stimulus between the proximal peripheral visual angles and 80 degree of peripheral eccentricity has a visual stimulus angle C of approximately 52.5 degree. Base on the formula above, the average visual stimulus angle C between the proximal peripheral visual angles and 80 degree of peripheral eccentricity is (30 degree*500 inch²+17.5 degree*500 inch²+52.5 degree*1000 inch²)/(2000 inch²)=(15000 degree*inch²+8750 degree*inch²+52500 degree*inch²)/(2000 inch²)=(76250 degree*inch²)/(2000 inch²)=38.125 degree.

The average visual stimulus angle of a visual stimulus located between the proximal peripheral visual angle B and 80 degree of peripheral eccentricity may be between approximately 0 to approximately 90 degree. Preferably, the average visual stimulus angle of the visual stimulus located between the proximal peripheral visual angles and 80 degree of peripheral eccentricity may be between approximately 0 to approximately 85 degree. More preferably, the average visual stimulus angle of the visual stimulus located between the proximal peripheral visual angles and 80 degree of peripheral eccentricity may be between approximately 0 to approximately 80 degree. More preferably, the average visual stimulus angle of the visual stimulus located between the proximal peripheral visual angles and 80 degree of peripheral eccentricity may be between approximately 0 to approximately 75 degree. More preferably, the average visual stimulus angle of the visual stimulus located between the proximal peripheral visual angles and 80 degree of peripheral eccentricity may be between approximately 0 to approximately 60 degree. More preferably, the average visual stimulus angle of the visual stimulus located between the proximal peripheral visual angles and 80 degree of peripheral eccentricity may be between approximately 0 to approximately 45 degree. More preferably, the average visual stimulus angle of the visual stimulus located between the proximal peripheral visual angles and 80 degree of peripheral eccentricity may be between approximately 0 to approximately 37 degree. More preferably, the average visual stimulus angle of the visual stimulus located between the proximal peripheral visual angles and 80 degree of peripheral eccentricity may be between approximately 12 to approximately 30 degree.

The visual stimulus or visual stimuli 102 comprising a plurality of contrast patterns 200 are presented to the peripheral vision in the peripheral visual fields 104, such that the visual stimuli 102 does not block the primary object 106. The primary object 106 is any object or image upon which the subject wishes to focus upon. For example, the primary object 106 can be any visual device such as monitors, computer screens, display device, television screens, movie screens, or any other device or object that the viewer intends to focus on such as natural sceneries, books, papers, notes, pictures, and the like.

The superior section(s) of a visual stimuli 102 is defined as a section(s) of the visual stimuli 102 that is(are) higher than the primary object 106. In some embodiments, the visual stimuli 102 comprising a plurality of contrast patterns 200 are presented to the peripheral vision in the peripheral visual fields 104, such that the superior section(s) of the visual stimuli 102 do not block the illumination of the superior section(s) of the visual stimuli by a light source such as lights on the ceiling, sun lights, or windows, etc. This may be implemented by using translucent or partially translucent visual stimuli, and/or increasing the visual stimuli angles of the superior sections of visual stimuli, and/or reducing the size of the superior sections of visual stimuli.

The visual stimuli 102 may be presented on a surface or a panel 118. For example, the panel 118 may be printed with various lines, shapes, designs, or other types of markings to create a plurality of contrast patterns 200.

In some embodiments, the visual stimuli 102 may be removably attached to the panels 118. Panels 118, visual stimuli 102, and contrast patterns 200 may be translucent, opaque, or variably translucent (transparent). In such embodiments, different visual stimuli 102 comprising different types of patterns and designs may be easily interchanged for one another depending on the viewer's needs and progression. Similarly, other viewers may view the same primary object with the same apparatus by merely replacing the visual stimuli 102, without significantly altering the panel 118.

In some embodiments, physical changes in the panel 118 may create contrast patterns 200. For example, changes in the texture of the panel 118 may create the contrast patterns 200. In some embodiments, as shown in FIG. 3A, the plurality of contrast patterns 200 are created by a depth contrast due to gradual or abrupt changes in depth between a first area and a second area. In some embodiments, a plurality of contrasts may be created by a differential in the reflection and/or translucency of the surroundings, thereby creating a luminance contrast. In some embodiments, the three dimensional structure of the panel 118 may create a depth contrast. For example, a plurality of protrusions or projections 300 and a plurality of depressions 302 on the panel 118 create changes in depth perception that can be deemed a contrast. In certain embodiments, the projections 300 and depressions 302 may be a plurality of ridges and a plurality of furrows. Such embodiments create an accordion-like or Japanese folding fan-like configuration. In embodiments comprising accordion-like configurations, the panel 118 may be constructed of pliable material so as to be adjustable. Thus, the panel 118 may be expanded and contracted like an accordion so that the size of the panel 118 is adjusted to suit the needs of the viewer or to fit on the primary object 106. The size or shape of the stimuli can be adjusted by cutting off one or more areas of the panel 118. For example, one may change the size and shape of the stimuli by cutting off area 350 along the dash line 352 as shown in FIG. 3C.

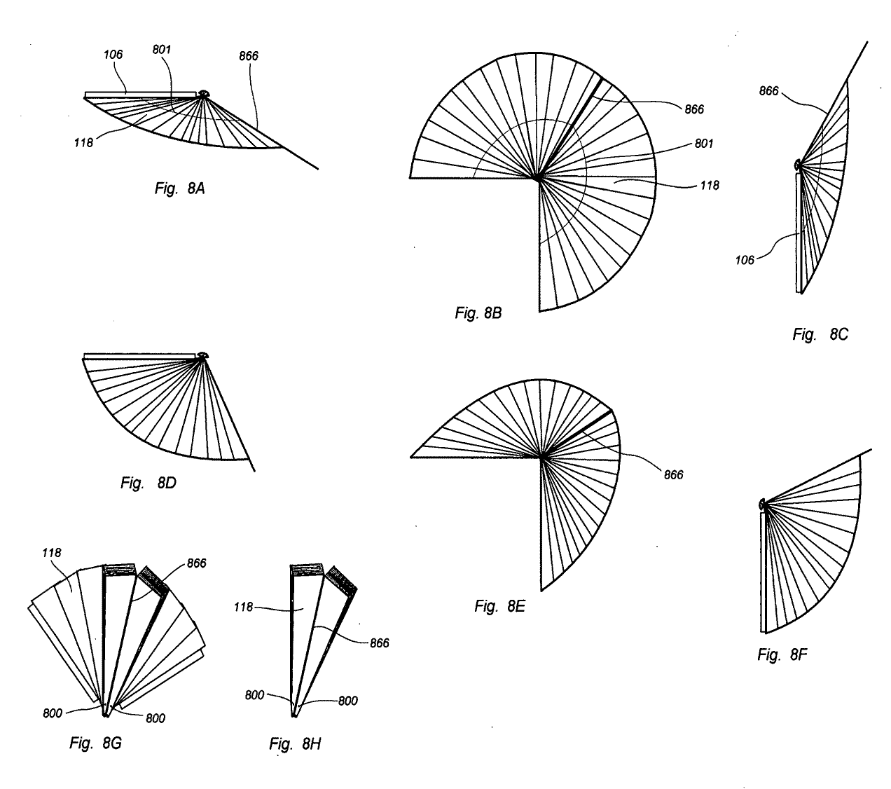

Other variations include a plurality of long rectangular, long trapezoid or long triangular panels 118 each comprising a first and second surface, wherein the first surface of a first panel abuts a second surface of a second panel. A plurality of panels 118 arranged in this orientation may be rotatably fastened together at a pivot point 800 at one end of the plurality of panels. This allows the plurality of panels to pivot along the pivot point to allow the plurality of panels to expand and collapse. The ranges of expansion maybe limited or controlled by lengths of strings, wires 801, or dimensions of papers, fabrics, sheets of pliable material, or the like, that connect each adjacent panel as shown in FIG. 8A and FIG. 8D.

Two of these collapsible type visual stimuli comprising a plurality of panels may be attached in series, or back-to-back as shown in FIGS. 8G and 8H. The attachment site 866 can be any fastening mechanism that would effectively form a hinge such that the angle between the two collapsible type visual stimuli is adjustable. This would allow for the adjustment of the distal peripheral visual angle A and the visual stimuli angle C.

In some embodiments, contrast patterns may be a combination of changes in the three dimensional structure of the panel 118 and designs that can be created, drawn, painted, illuminated on the panel and the like.

In some embodiments, the panel 118 may further comprise a first support member 304 and a second support member 305, each with fastening mechanisms 306 so as to provide structural stability and be removably attached to the primary object. For example, magnets, adhesives, snap buttons or hook and loop type fastening mechanisms, such as those sold under the trademark VELCRO® may be utilized to removably attach the panel 118 to the primary visual object 106.

Many versions of this invention have been contemplated. A few examples are described herein without limitation.

In one embodiment, as shown in FIGS. 3A-3E, a collapsible vision correction device may be created by taking a rectangular panel 118 having a first side 308 and a second side 310 and creating a plurality of folds 300, 302 in a longitudinal direction parallel to the first side 308 and the second side 310 so as to create a plurality of ridges and furrows. A rectangular or square shaped corner piece 312 may then be cut out from the first side 308, thereby exposing a third side 314 and a fourth side 316. In such an embodiment, the rectangular cut-out is shaped to fit the corner of a monitor, screen, or television. The first side 308 and fourth side 316 may comprise first support member 304 and second support member 305, respectively, to attach the panel 118 to a visual display such as a monitor, screen or television. The first side 308 may be pulled to elongate the panel in a first direction, then the third side 314 may be attached to the visual display. The second side 310 may then be pulled to elongate the panel in a second direction. A first extension bar 318 may be rotatably connected to the panel 118 or the second support member 305 at or near the junction where the third side 314 and the fourth side 316 meet. The first extension bar 318 may be a rigid or bendable structure that is attachable to the second support member 305 or the panel 118 to keep the panel 118 in an extended configuration. A bendable first extension bar 318 allows a portion of the panel 118, when in the extended configuration, to have an adjustable distal peripheral visual angle A (or adjustable distal peripheral visual angles) and an adjustable visual stimuli angle C (or adjustable distal visual angles), an adjustable average visual stimulus angle, and adjustable distances. The support members 304, 305 and first extension bar 318 may be attachable to other structures by fastening mechanisms 306 that allow for quick and easy attachment and detachment, such as the hook and loop, buttons and holes, adhesives, or magnets.

To disassemble the panel 318 from the visual display, the support members 304, 305 and first extension bar 318 may be detached and the panel 118 collapsed like an accordion along the plurality of folds 300, 302.

As shown in FIG. 3B, in another embodiment of the vision correction device, a portion of the panel 118 may be bent at a first midpoint 320 along the second side 310 such that a first end surface 322 and a second end surface 324 abut one another. The first midpoint 320 is located midway between the first end surface 322 and the second end surface 324. The first end surface 322 and the second end surface 324 may be fastened together with, for example, an adhesive. This converts the second side 310 into a radial panel with a plurality of contrast patterns created by the plurality of folds. The left over plurality of folds will creates a midsection 326 between the first side 308 and the second side 310, which can be used to expand panel 118. In other embodiments, the midpoint 320 can be implemented using bookbinding method, such as spiral binding or coil binding, sewing through the fold, or the double-fan adhesive binding method, etc, at mid point 320 to adhere (attach) the stack of folded panel to each other at the pivot point or mid point 320, or to adhere (attach) the stack of folded panel to a soft bendable material like the spine of a book at mid point 320.

The midsection 326 allows for expansion in the second direction, if desired. Thus, the foldable, compact panel can be attached to the visual display at the third and fourth sides 314, 316. The midsection 326 may be expanded if desired. A second extension bar 319 may be attached along the first end surface 322 and second end surface 324 junction or along any fold on the second side 310. The second extension bar 319 may have a fastening mechanism 332 to connect with the first extension bar 318, thereby allowing the midsection 326 to be fully or partially extended or not extended at all as shown in FIG. 3D. The first extension bar 318 may be bendable, such that, when attached to the second extension bar 319 in the extended configuration a portion of the panel 118 has adjustable distal peripheral visual angles A and an adjustable visual stimuli angles C.

To store this device, the panel 118 is detached from the visual display and the first extension bar 318 is detached from the second extension bar 319. The first extension bar 318 may be rotated downward towards the second support member 305. The first side 308 may be collapsed and the midsection 326 may be collapsed if previously expanded. The fourth side 316 can be rotated circularly about a second midpoint 328, located where the third side 314 and the fourth side 316 intersect, towards the second side 310, then back towards the first side 308. The fourth side 316 can then be removably secured to the first side 308 via the first and second support members 304 and 305, as shown in FIG. 3E.

Two of these collapsible vision correction devices may be assembled as mirror images to attach to the opposite side of the visual display or primary object. Alternatively, the two collapsible vision correction devices may be assembled as a single unit.

In another embodiment, the first support member 304 is directly attached to the primary object, such as attach along the top edge of a screen, instead of attached to the “mirror image” collapsible panel's support member 304, and form an fan like (or ¼ of a circle) shape instead of the rectangular shaped for the section that is directly above the screen as shown in FIGS. 3Fa, 3Fb and 3G. In another embodiment, the rectangular or square shaped corner piece 312 is not cut out from panel 118 and the second support member 305 is directly attached to the portion of edge 308 that is located just below support member 304. As shown in FIG. 3G through 3L, the medial tip of first support member 304, which is near the center of edge 308, is rotatably connected to the top tip of the third support member 307. Similarly, medial tip of the second support member 305, which is near the center of edge 308, is rotatably connected to the bottom tip of the third support member 307. As shown in FIG. 3G, the first support member 304 is directly attached to the top edge of the primary object 106, such as attach along the top edge of a screen, while the second support member is directly attached to the side of the primary object 106.

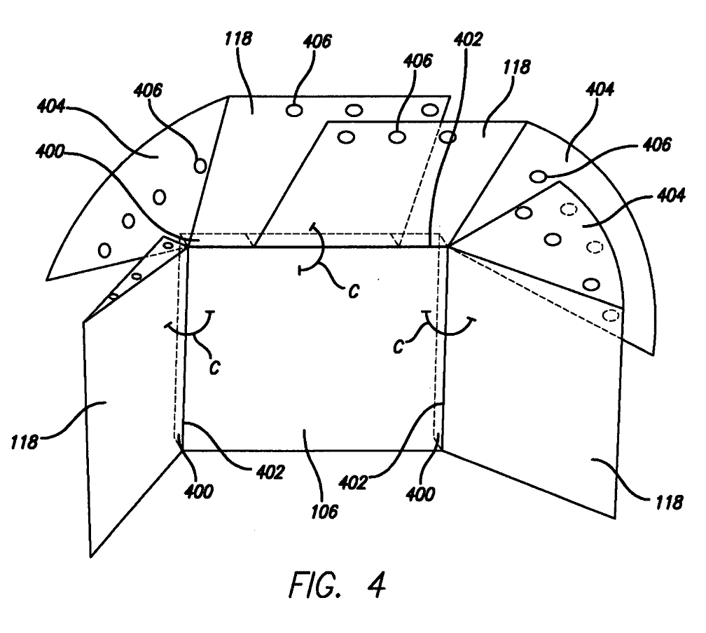

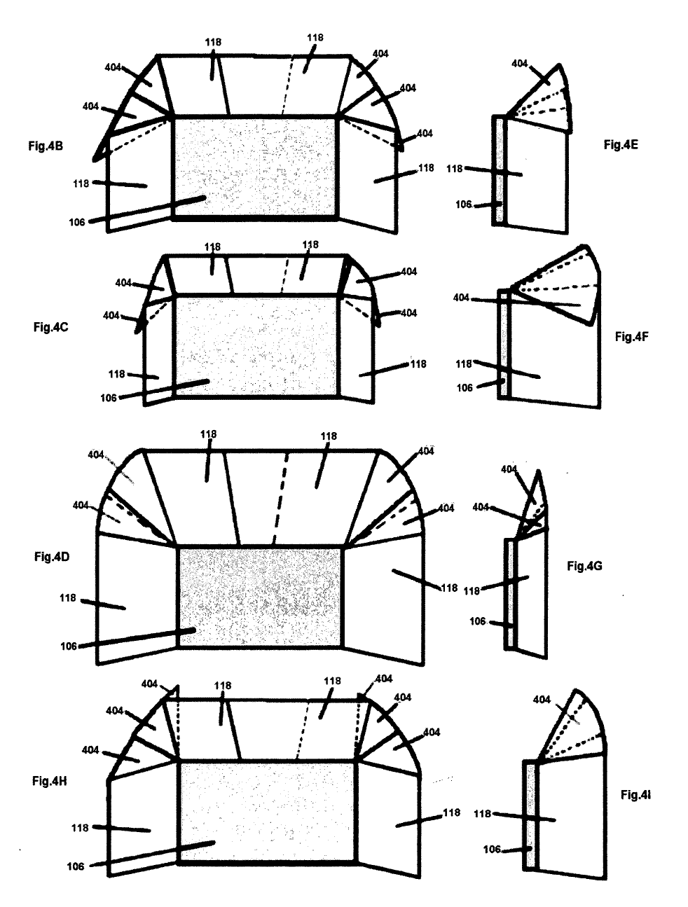

As shown in FIG. 4, in some embodiments, the vision correction device may comprise a plurality of panels 118. The panels 118 may comprise a plurality of attachments sites 400 for attachment to the primary object 106 or to each other. Fasteners such as buttons, magnets, hook and loop, tabs, and the like may be used for quick attachment and release. Also, the panels 118 may be pliable or the panels may have movable joint sections 402, thereby allowing the visual stimuli angles, average visual stimulus angle, distal peripheral visual angles of each panel 118, and the distances from the eyes to different areas of the panel 118 to be adjustable.

Each panel 118 may further comprise flaps 404. These flaps 404 may further facilitate adjustability of the distal peripheral visual angles and visual stimuli angles as well as increase the overall surface area of the vision correction device. In addition, the flaps 404 may be folded into the main portion of the panel 118 so that the panels 118 can be folded into a compact travel size.

One bendable rod or plurality of bendable rods may be further attached to each panel 118 and flap 404, there by allowing visual stimuli angles, average visual stimulus angle, distal peripheral visual angles of each panel 118, and the distances from the eyes to different areas of each panel 118 to be adjustable.

In some embodiments, the flaps 404 may be rotatably or slidably coupled to the panel 118 so that the flaps 404 can slide along the face of the panel 118. This sliding action further facilitates the expansion of the surface area of the panel as well as the disassembly for compact storage. The panels 118 may further comprise fastening mechanisms 406 to secure the panels at a desired distance and at a desired distal peripheral visual angles, proximal peripheral visual angles and visual stimuli angles, and allow panels 118 to be fitted on to different primary objects 106 of a variety of sizes. Fastening mechanisms such as magnets, buttons, hook and loop, tabs and the like, may be used for quick attachment and release.

The left superior panel and the right lateral panel may be swapped in positions, and the left lateral panel and the right superior panel may be swapped in positions as shown in FIG. 4H and FIG. 4I.

In certain embodiments, the panel 118 may comprise a fastening mechanism to attach to the primary object 106. In some embodiments, the panel 118 comprises a support member or a frame such that the panel is free standing. In some embodiments, the panel 118 further comprises a pocket to hold various items.

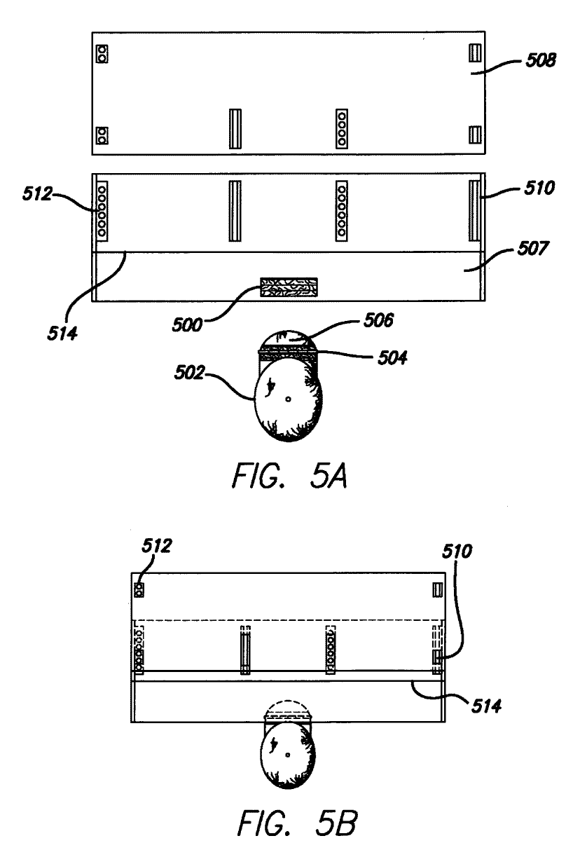

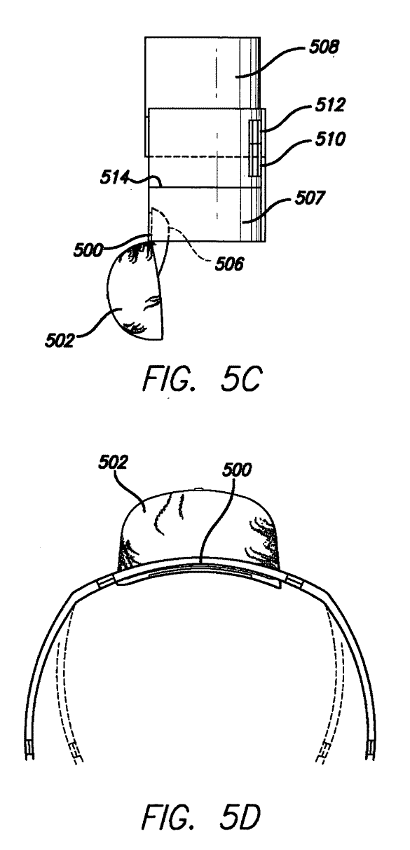

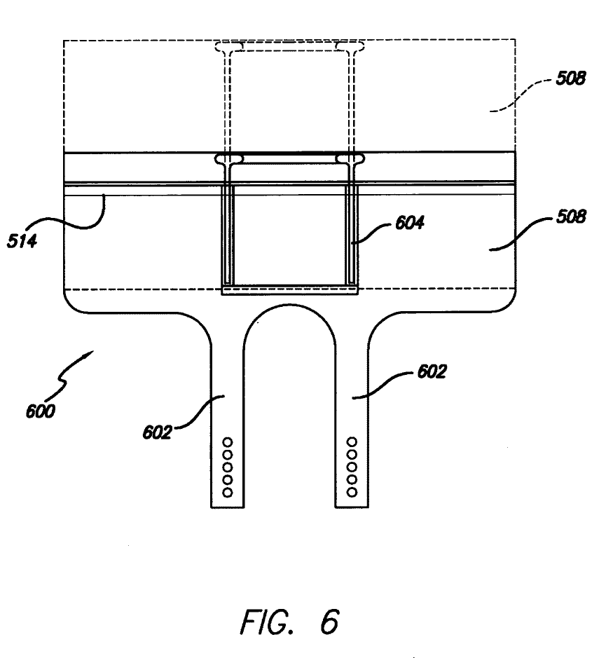

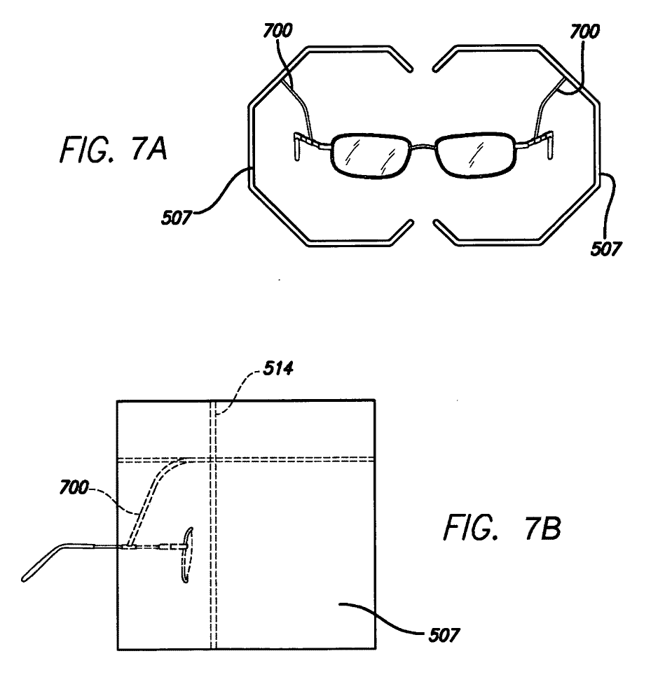

As shown in FIGS. 5-6, the vision correction device may be wearable so that the user can wear the device, for example, as a modified hat, attached to a standard hat, attached to a pair of eyeglasses, or the like. In certain embodiments, the vision correction device comprises a single pliable panel 507 with an attachment site 500. A standard hat 502, such as a baseball cap may comprise a reciprocal attachment site 504 on the top side of the brim 506. The panel 507 may be attached to the brim 506 such that the sides of the panel hang down far enough to provide the visual stimulus 102 to the peripheral visual field 104 as shown in FIGS. 5C and 5D. The reciprocal attachment site 504 may contain a hinge to allow easy “flipping” of the panel 118. The pliable panel 507 may further comprise a bendable rod or guide wire 514. The guide wire 514 may be bendable and provides structural stability to the panels 507. As shown in FIG. 5D, the guide wire 514 allows the panel 507 to maintain any bent position such that the panels may be placed at a predetermined position or a predetermined angle relative to the central visual axis 108.

In another embodiment, the vision correction device may comprise at least two panels 507, 508. The first panel 507 may be attached to the brim 506 of the hat 502. The second panel 508 may be movably, slidably, or rotatably attached to the first panel 507. This provides a telescoping effect to increase the total surface area of the visual stimulus 102 presented to the peripheral visual field 104. The second panel 508 may be removably attached to the first panel 507 using a hook and loop system, such as VELCRO®, buttons 512, tabs, magnets and the like. Alternatively, the second panel 508 may be slidably attached to the first panel 507 using a rod and sleeve 604 or tongue and groove system 510 or the like. In some embodiments, the second panel 508 may be rotatably attached to one attachment site with a button, tab or the like and removably attached to a second site to prevent rotation.

In another embodiment, as shown in FIG. 6, the vision correction device 600 may be adapted to be worn directly on the head instead of attached to a hat. For example, the vision correction device may comprise a head loop 602 that can be wrapped around and fastened to the head.

In another embodiment, the vision correction device may further comprise an adjustable member 700 connecting the panel 507 to the wearable article, such as a hat or glasses, to allow the panel 507 to move in all directions relative to the central visual axis 108. The adjustable member 700 may be, for example, a flexible rod, bendable rod, a hinge, or the like, such that the distal peripheral visual angles, the proximal peripheral visual angles, the visual stimulus angles and distances are adjustable. Flexible rods may be comprised of multiple segments connected by resistive or lockable joints. Each segment may be positioned in various angles and orientations and held in place due to the resistance in the joints or by locking the joints in place. In another embodiment, the rods may be made of bendable wires, thus eliminating the need of separate segments and joints.

This invention is also directed towards a method for treating vision disorders, comprising providing contrast patterns 200, to a peripheral vision, thereby treating and/or preventing vision disorders.

The method further comprises the steps of positioning the visual stimuli 102 relative to the central visual axis 108 without blocking the primary object 106, such that the proximal peripheral visual angle(s) B is/are between approximately zero degree and approximately 70 degree, and the distal peripheral visual angle(s) A is/are between proximal peripheral visual angles and up to approximately 150 degree of peripheral eccentricity. In another embodiment, the proximal peripheral visual angle(s) B is/are approximately between 10 and 30 degree, and the distal peripheral visual angle(s) A is/are between approximately 60 degree and up to approximately 150 degree of peripheral eccentricity. In addition, the visual stimuli 102 may be positioned at predetermined distances from an eye. Preferably, the distance from any part of a visual stimulus to the center of pupil of an eye is at least 7 cm. More preferably, the distances from the center of pupil of an eye to a visual stimulus is at least 10 cm.

While the present invention has been described in regard to particular embodiments, it is recognized that additional variations of the present invention may be devised without departing from the inventive concept.

### INDUSTRIAL APPLICABILITY

This invention may be industrially applied to devices that prevent and/or treat eye disorders, in particular, vision disorders.

### APPENDIX

Attached in the appendix are additional background information regarding converting 3D orthogonal (x, y, z) coordinates to spherical (q, e, d) coordinates, background information regarding spatial frequency spectral analysis, and disclosures incorporated herein.

### APPENDIX

### Background Information on Converting 3D (x, y, z) Orthogonal Coordinates to Spherical (q, e, d) Coordinates

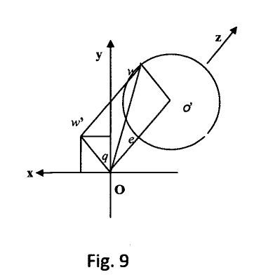

In FIG. 9, the drawing shows the 3-D x, y, z coordinate, and the spherical q, e, d, coordinate, where x is the horizontal axis, y is the vertical axis, z is the central visual axis 108 as shown in FIGS. 1A and 1B, and q is axle deviation, e is peripheral eccentricity, and d is the distance from a coordinate to the origin where axis x, y, z intersect or the central visual point 110 as show in FIGS. 1A and 1B.

In FIG. 9, the point w′ is the orthogonal projection from a point w with coordinate (x, y, z) onto the x, y-axle plane, and has a coordinate of (x, y, 0) in the x, y, z 3-D space. Point o′ is the orthogonal projection from the point w onto the z-axis, and has a coordinate of (0, 0, z) in the x, y, z 3-D space. The plane defined by (origin, w, w′ and o′) is orthogonal to the x, y-axle plane.

Axle deviation, or q, is the counter-clockwise angle between the y-axis and the ray from origin to the point w′. Peripheral eccentricity, or e, is the angle between z-axis and the ray from origin to the point w on the (origin, w, w′ and o′) plane. Distance, d, is the distance between the origin and the point w.

The axle deviation, q, of point w(x, y, z) is defined as the following:

Range: 0≦q<2π

If x=0, y=0, then q=0;

If y=0, x>0, then q=π/2;

If y=0, x<0, then q=3*π/2;

If y>0 and x>0, then q=arctan(x/y);

If y>0 and x<0, then q=arctan(x/y)+3*π/2;

If y<0, then q=arctan(x/y)+π;

The peripheral eccentricity, e, of point w(x, y, z) is defined as the following:

Range: 0≦e≦π

If z=0, then e=π/2;

If z>0, then e=arctan(((x̂2+ŷ2)̂0.5)/z );

If z<0, then e=arctan(((x\hat{2}+ŷ2)̂0.5)/z)+π;

The distance, d, from origin to point w(x, y, z) is defined as the following:

Range: d≧0

d=(x\hat{2}+y\hat{2}+ẑ2)̂0.5;

In another word, the point w has a coordinate of (x, y, z) in the 3-D (x, y, z) orthogonal coordinate system, and a coordinate of (q, e, d) in the spherical (q, e, d) coordinate system.

Note: All peripheral eccentricities and axle deviations referred by this patent application are calculated by the method provide above.

Background Information Regarding Spatial Frequency Spectral Analysis

The spatial frequency contrast function perceived by the retina from a simple or complex surface (not necessarily a plane or smooth surface) can be defined in 2-D axial coordinates by f (x, y) or in spherical coordinates by f (q, e), where e is the peripheral eccentricity and q is the axle deviation. A spatial frequency contrast function f(x, y) can be (approximately) transformed to function F(X, Y) in the frequency domain. This transformation can be done via Fourier transform (frequency spectrum analysis). The spatial frequency contrast function f (q, e), can also be (approximately) transformed to function F(Q, E) in the frequency domain. F(Q, E) have unit of cycles per degree of peripheral eccentricity and cycles per degree of axis deviation. This frequency spectral analysis can be approximated using digital Fourier transform approximation in Matlab©.

The total accommodation stimulus is insufficient because of lack of accommodation stimulus from the peripheral visual field(s) during nearwork such as reading books, staring into a visual display monitor, focus on other materials such as books, notes, papers, and the like without using the said method. The principle mechanism of the said method is to provide additional accommodation stimuli in the peripheral visual field(s) in order to increase the accommodation of the lens of eye(s) and reduce accommodation lag. The effect of the said method can be demonstrated by measuring changes of the choroid plexus thickness with and without the said apparatus over 10 to 14 days.

What is claimed:

1. A portable apparatus for preventing, controlling and/or treating a vision disorder or vision disorders, comprising: a visual stimulus or a plurality of visual stimuli provided to a peripheral visual field or peripheral visual fields comprising a contrast pattern or contrast patterns, such that the visual stimulus or visual stimuli is/are placed adjacent to a primary object,
(i) wherein a proximal peripheral visual angle is defined by a ray directed towards a point on the proximal end of the visual stimulus, a central visual point, and a central visual axis; a distal peripheral visual angle is defined by a ray directed towards a distal end of the visual stimulus, a central visual point, and a central visual axis;
(ii) wherein superior section(s) of visual stimuli is (are) defined as section(s) of visual stimulus (stimuli) that is (are) located higher than the primary object;
(iii) wherein a contrast pattern is selected from the group consisting of:
(a) a luminance contrast,
(b) a translucency/transparency contrast,
(c) a (color) spectral contrast, and
(d) a depth contrast;
(iv) wherein the apparatus is perceived by the eye(s) as approximately not completely behind the primary object;
(v) wherein the relevant spatial frequencies of a contrast pattern (or contrast patterns) are spatial frequencies between approximately 0.1 and approximately 10 cycles per degree in any direction;
(vi) wherein the standard deviation of the relative luminance of a contrast pattern (or contrast patterns) that has been band pass filtered for spatial frequency between approximately 0.1 and approximately 10 cycles per degree, is at least approximately 2.5%; and when the standard deviation of the relative luminance of a contrast pattern (or contrast patterns) that has been band pass filtered for spatial frequency between approximately 0.1 and approximately 10 cycles per degree, is less than approximately 2.5%, the relative-luminance-contrast is at least approximately 10%.
(vii) wherein the mean relative luminance of the contrast pattern(s) is at least approximately 5%.
(viii) wherein the distance from any part of a visual stimulus to the center of pupil of an eye is at least approximately 7 cm.

2. The apparatus of claim 1, wherein one or more areas of the contrast pattern comprises a spatial frequency spectral analysis profile defined by the equation: X/(1+10*e) with units of cycle per degree of peripheral eccentricity and/or cycle per degree of axle deviation, where X is a function, a number, or numbers from approximately 0.5 to approximately 10 and e is angle of peripheral eccentricity expressed in radian unit, or degree of peripheral eccentricity *π/180.

3. The apparatus of claim 1, wherein the superior section(s) of the visual stimulus (stimuli) is (are) semi-translucent (or transparent), partially translucent (or transparent), or completely translucent (or transparent), in order to partially block, or not block the illumination of the primary object and/or the said superior section(s) of the visual stimulus (or visual stimuli) from light sources such as ceiling light(s), lights from window(s), sun lights, or the alike.

4. The apparatus of claim 1, wherein the superior section(s) of the visual stimulus (stimuli) is (are) opaque in order to block light from a ceiling light(s), light from window(s), sun light, or the alike.

5. The apparatus of claim 1, wherein the visual stimulus (or visual stimuli) does (do) not have a superior section or (superior sections).

6. The apparatus of claim 1, wherein, the standard deviation of the relative luminance of contrast pattern(s) that has been band pass filtered for spatial frequency between approximately 0.1 and approximately 10 cycles per degree, is at least approximately 5%.

7. The apparatus of claim 1, wherein, the standard deviation of the relative luminance of contrast pattern(s) that has been band pass filtered for spatial frequency between approximately 0.1 and approximately 10 cycles per degree, is at least approximately 7.5%.

8. The apparatus of claim 1, wherein, the standard deviation of the relative luminance of contrast pattern(s) that has been band pass filtered for spatial frequency between approximately 0.1 and approximately 10 cycles per degree, is at least approximately 10%.

9. The apparatus of claim 1, wherein, the standard deviation of the relative luminance of contrast pattern(s) that has been band pass filtered for spatial frequency between approximately 0.1 and approximately 10 cycles per degree, is at least approximately 12.5%.

10. The apparatus of claim 1, wherein when the standard deviation of relative luminance of the relevant spatial frequency of contrast pattern(s) is less than approximately 3.5%, the relative-luminance-contrast is at least approximately 17%.

11. The apparatus of claim 1, wherein when the (color) spectral contrast is used, the difference between the one set and another set of frequencies of the color spectral contrast is at least approximately 15 nanometers.

12. The apparatus of claim 1, wherein the contrast pattern(s) or portion(s) of the contrast pattern(s) is (are) created from different grades of translucencies or opaqueness.

13. The apparatus of claim 1, wherein the contrast pattern(s) or portion(s) of the contrast pattern(s) is (are) generated by a light source, light sources, a light projection or light projections.

14. The apparatus of claim 1, wherein the proximal peripheral visual angle(s) is/are between approximately 0 degree and up to approximately 80 degree.

15. The apparatus of claim 1, wherein the distal peripheral visual angle(s) is/are between the proximal peripheral visual angles and up to approximately 150 degree.

16. The apparatus of claim 1, wherein the visual stimuli angle(s) is/are between approximately −90 degree and approximately 270 degree.

17. The apparatus of claim 1, wherein the average visual stimulus angle of the visual stimulus is between approximately 0 degree and approximately 90 degree.

18. The apparatus of claim 1, wherein the average visual stimulus angle of the visual stimulus located between the proximal peripheral visual angles and approximately 80 degree of peripheral eccentricity is between approximately 0 degree and approximately 90 degree.

19. The apparatus of claim 1, wherein the distance from any part of a visual stimulus to the center of pupil of an eye is at least approximately 10 cm.

20. The apparatus of claim 1, wherein the visual stimulus further comprises a fastening member or fasting members to attach to the primary object.

21. The apparatus of claim 20, where in the fastening member (or fasting members) is (are) selected from the group consisting of magnet, loop and hook, button, button and hole, adhesive, or the like.

22. The apparatus of claim 1, wherein the visual stimulus (stimuli) further comprises (comprise) a support member or support members such that the apparatus is portable and/or free standing.

23. The apparatus of claim 1, wherein the visual stimulus further comprises a pocket or pockets to hold an item or items.

24. The apparatus of claim 1, wherein the visual stimulus further comprising adjustable member(s) that is (are) attachable to the said visual stimulus and/or other visual stimulus or visual stimuli to allow the proximal peripheral visual angles to be adjustable between approximately zero degree and approximately 80 degree.

25. The apparatus of claim 1, wherein the visual stimulus further comprising adjustable member(s) to allow the distal peripheral visual angles to be adjustable between the proximal peripheral visual angles up to approximately 150 degree.

26. The apparatus of claim 1, wherein the visual stimulus further comprising adjustable member(s) to allow the visual stimuli angles to be adjustable between approximately −90 degree and approximately 270 degree.

27. The apparatus of claim 1, wherein the visual stimulus further comprising adjustable member(s) to allow the visual stimuli angle to be folded behind or folded in front of the primary object for storage.

28. The apparatus of claim 1, wherein the visual stimulus further comprising adjustable member(s) to allow the average visual stimuli angle of the visual stimulus to be adjustable between approximately 0 degree and approximately 90 degree.

29. The apparatus of claim 1, wherein the visual stimulus further comprising adjustable member(s) to allow the average visual stimulus angle of the visual stimulus located between the proximal peripheral visual angles and approximately 80 degree of peripheral eccentricity to be adjustable between approximately 0 degree and approximately 90 degree.

30. The apparatus of claim 24-29, wherein the adjustable member(s) is (are) a bendable and/or flexible rod(s), telescoping tube(s), or bendable plate(s), or non-bendable rod/plates connected by adjustable joints/hinges, or the alike.

31. The apparatus of claim 1, wherein the visual stimulus is a panel or plurality of panels comprising a contrast pattern or plurality of contrast patterns.

32. The apparatus of claim 31, wherein the contrast pattern(s) is (are) printed on the panel;

33. The apparatus of claim 31, wherein the contrast pattern(s) is (are) attached to the panel.

34. The apparatus of claim 31, wherein the contrast pattern(s) is (are) created by a plurality of projections and a plurality of depressions on the panel.

35. The apparatus of claim 31, wherein the contrast pattern(s) is (are) created by a plurality of ridges and a plurality of furrows.

36. The apparatus of claim 31, wherein the panel comprises a means for folding the panel into a compact structure.

37. The apparatus of claim 31, wherein the panel further comprises a fastening member or fasting members to attach to the primary object.

38. The apparatus of claim 31, wherein the panel further comprises a support member such that the panel is free standing.

39. An apparatus for preventing and/or treating a vision disorder, comprising:
(i) a visual stimulus or visual stimuli provided to a peripheral visual field or peripheral visual fields comprising a contrast pattern or a plurality of contrast patterns;
(a) wherein the contrast pattern(s) is/are selected from the group consisting of:
(A) a luminance contrast,
(B) a translucency/transparency contrast,
(C) a (color) spectral contrast, and
(D) a depth contrast,
(b) wherein the relevant spatial frequencies of a contrast pattern is between approximately 0.1 and approximately 10 cycles per degree of spatial frequencies;
(c) wherein, the standard deviation of the relative luminance of contrast pattern(s) that has been band pass filtered for spatial frequency between approximately 0.1 and approximately 10 cycles per degree, is at least approximately 5%;
(d) wherein the superior sections of visual stimuli are defined as sections of visual stimuli that is located higher than the primary object;
(ii) a wearable device supporting the visual stimulus such that the contrast pattern or plurality of contrast patterns are perceived by a peripheral vision.

40. The apparatus of claim 39, wherein the distance from any part of a visual stimulus to the center of pupil of an eye is at least approximately 7 cm. preferably, the distance from any part of a visual stimulus to the center of pupil of an eye is at least approximately 10 cm.

41. The apparatus of claim 39, wherein the contrast pattern comprises a spatial frequency spectral analysis profile defined by the equation: X/(1+10*e) with units of cycle per degree of peripheral eccentricity and/or cycle per degree of axle deviation, where X is a function, or a number, or numbers from approximately 0.5 to approximately 10 and e is angle of peripheral eccentricity expressed in radian unit, or degree of peripheral eccentricity *π/180.

42. The apparatus of claim 39, wherein the luminance contrast ranges from approximately 10% to approximately 100%.

43. The apparatus of claim 39, wherein the standard deviation of relative luminance of the relevant spatial frequencies of contrast pattern(s) is at least approximately 7.5%, preferably at least approximately 10%, more preferably at least approximately 12.5%, more preferably at least approximately 15%.

44. The apparatus of claim 39, wherein when the standard deviation of relative luminance of the relevant spatial frequency of contrast pattern(s) is less than approximately 5%, the relative-luminance-contrast is at least approximately 20%.

45. The apparatus of claim 39, wherein when the (color) spectral contrast is used, the difference between the one set and another set of frequencies of the color spectral contrast is at least approximately 15 nanometers.

46. The apparatus of claim 39, wherein the contrast pattern(s) or portion(s) of the contrast pattern is (are) created from different grades of translucencies or opaqueness.

47. The apparatus of claim 39, wherein the contrast pattern(s) or portion(s) of the contrast pattern(s) is (are) generated by a light source, light sources, a light projection or light projections.

48. The apparatus of claim 39, further comprising an adjustable member or adjustable members attached to the visual stimulus or visual stimuli to allow the visual stimulus or visual stimuli to move relative to the central visual axis.

49. The apparatus of claim 48, wherein the adjustable member(s) is (are) a bendable and/or flexible rod(s), telescoping tube(s), or hinge(s) such that one or more of the following angle(s) and/or distance(s) are adjustable:
(a) distance(s) from the visual stimuli to the eye(s),
(b) proximal peripheral visual angle(s),
(c) distal peripheral visual angle(s),
(d) visual stimuli angle(s), and
(e) average visual stimuli angle(s).

50. The apparatus of claim 39, wherein the visual stimulus is a panel or plurality of panels comprising a contrast pattern or plurality of contrast patterns.

51. The apparatus of claim 39, wherein the visual stimulus is provided on a first flexible panel and this flexible panel is removably attached to the wearable device or hinge(s) on the wearable device to allow visual stimulus (stimuli) to be flipping up or down.

52. The apparatus of claim 39, further comprising a second flexible panel comprising a visual stimulus, movably attached to the first flexible panel and allow the proximal peripheral visual angles to be adjustable.

53. The apparatus of claim 39, wherein the superior section(s) of the visual stimulus (stimuli) is (are) semi-translucent (or transparent), partially translucent (or transparent), or completely translucent (or transparent), in order to partially block, or not block the illumination of the primary object and/or the said superior section(s) of the visual stimulus (or visual stimuli) from light sources such as ceiling light(s), lights from window(s), sun lights, or the alike.

54. The apparatus of claim 39, wherein the superior section(s) of the visual stimulus (stimuli) is (are) opaque in order to block light from a ceiling light(s), light from window(s), sun light, or the alike.

55. A method for preventing, controlling, and/or treating a vision irregularity or vision irregularities, comprising a visual stimulus or visual stimuli provided to a peripheral visual field or peripheral visual fields, wherein the visual stimulus (stimuli) comprise of a contrast pattern or a plurality of contrast patterns, thereby treating and/or preventing the vision irregularity or irregularities such as eye fatigue, and myopia,
(i) wherein the contrast pattern(s) is (are) selected from the group consisting of:
(a) a luminance contrast,
(b) a translucency/transparency contrast,
(c) a (color) spectral contrast, and
(d) a depth contrast;
(ii) wherein the contrast pattern(s) is (are) perceived by the eye(s) as approximately not completely behind the primary object;
(iii) wherein the contrast pattern(s) does (do) not block the center gaze of the eye(s);
(iv) wherein the relevant spatial frequencies of a contrast pattern are spatial frequency between approximately 0.1 and approximately 10 cycles per degree in any direction;
(v) wherein the standard deviation of the relative luminance of contrast pattern(s) that has been band pass filtered for spatial frequency between approximately 0.1 and approximately 10 cycles per degree, is at least approximately 3.5%;
(vi) wherein the mean relative luminance of the contrast pattern(s) is at least approximately 7%.

56. The method of claim 55, wherein the contrast pattern comprises a spatial frequency spectral analysis profile defined by the equation: X/(1+10*e) with units of cycle per degree of peripheral eccentricity and/or cycle per degree of axle deviation, where X is a function, or a number, or numbers from approximately 0.5 to approximately 10 and e is angle of peripheral eccentricity expressed in radian unit, or degree of peripheral eccentricity *π/180.

57. The method of claim 55, wherein the luminance contrast ranges from approximately 10% to approximately 100%.

58. The method of claim 55, wherein the standard deviation of relative luminance of the relevant spatial frequencies of contrast pattern(s) is at least approximately 10%, preferably at least approximately 12.5%, more preferably at least approximately 15%, more preferably at least approximately 20%.

59. The method of claim 55, wherein when the standard deviation of relative luminance of the relevant spatial frequency of contrast pattern(s) is less than approximately 7%, the relative-luminance-contrast is at least approximately 17%.

60. The method of claim 55, wherein when the (color) spectral contrast is used, the difference between the one set and another set of frequencies of the color spectral contrast is at least approximately 15 nanometers.

61. The method of claim 55, wherein the contrast pattern(s) or portion(s) of the contrast pattern(s) is (are) created from different grades of translucencies or opaqueness.

62. The method of claim 55, wherein the contrast pattern(s) or portion(s) of the contrast pattern(s) is (are) generated by a light source, light sources, a light projection or light projections.

63. The method of claim 55, wherein the visual stimulus (or visual stimuli) is (are) next to the primary object.

64. The method of claim 55, wherein the majority of distal peripheral visual angles of the visual stimuli in left and right peripheral visual fields is greater than or equal to approximately 30 degree of peripheral eccentricity.

65. The method of claim 55, wherein the average visual stimulus angle of the visual stimulus is between approximately 0 degree and approximately 90 degree, preferably the average visual stimulus angle of the visual stimulus (visual stimuli) is (are) less than or equal to approximately 45 degree, more preferably the average visual stimulus angle of the visual stimulus (visual stimuli) is (are) less than or equal to approximately 30 degree.

66. The method of claim 55, wherein the distance from any part of a visual stimulus to the center of pupil of an eye is at least approximately 7 cm.

67. The method of claim 55 further comprises the step(s) of adjusting the proximal peripheral visual angle between approximately zero degree and approximately 80 degree.

68. The method of claim 55 further comprises the step(s) of adjusting the distal peripheral visual angles between the proximal peripheral visual angles up to approximately 150 degree.

69. The method of claim 55 further comprises the step(s) of adjusting the visual stimuli angle(s).

70. The method of claim 55 further comprises the step(s) of adjusting the average visual stimuli angle of the visual stimulus to be between approximately 0 degree and approximately 90 degree to achieve optimal balance between comforts and reducing accommodation lags of the eyes.

71. The method of claim 55 further comprising the step of positioning the contrast pattern relative to a central visual axis without blocking a center of gaze, such that the visual stimulus or visual stimuli occupy from as low as approximately zero degree up to approximately 150 degree of peripheral eccentricities.

72. The method of claim 55, further comprising the steps of positioning the contrast stimuli at a predetermined distance from an eye, wherein the ratio of distance from any part of a visual stimulus to the center of pupil of an eye vs. the distance between the primary object and the eye is less than or equal to approximately 1, and the distance from any part of a visual stimulus to the center of pupil of an eye is greater than or equal to approximately 7 cm.

73. The method of claim 55, wherein the distance from any part of a visual stimulus to the center of pupil of an eye is greater than or equal to approximately 10 cm and less than the distance between the primary object and the eye.

74. A method for preventing, controlling, and/or treating a vision irregularity or vision irregularities, comprising:
(i) providing a contrast pattern or contrast patterns (the visual stimuli) to a peripheral visual field or peripheral visual fields, wherein the contrast pattern(s) is (are) selected from the group consisting of a luminance contrast, a translucency/transparency contrast, a spectral contrast, and a depth contrast; and
(a) wherein the contrast pattern(s) comprises a spectral analysis profile defined by the equation: X/(1+10*e) with units of cycle per degree of peripheral eccentricity and/or cycle per degree of axle deviation, where X is a function, or a number, or numbers from approximately 0.5 to approximately 10 and e is angle of peripheral eccentricity expressed in radian unit, or degree of peripheral eccentricity *π/180;
(b) wherein the contrast pattern(s) is (are) perceived by the eye(s) as approximately not completely behind the primary object;
(c) wherein the relevant spatial frequencies of a contrast pattern are spatial frequencies between approximately 0.1 and approximately 10 cycles per degree in any direction;
(d) wherein the standard deviation of the relative luminance of a contrast pattern (or contrast patterns) that has been band pass filtered for spatial frequency between approximately 0.1 and approximately 10 cycles per 34 degree, is at least approximately 2.5%;
(ii) positioning the contrast pattern(s) relative to a central visual axis without blocking a center of gaze, such that the contrast pattern(s) (or visual stimuli) occupies from as low as approximately zero degree up to approximately 150 degree;
(iii) adjusting the average visual stimulus (stimuli) angle(s) between approximately zero degree and up to approximately 90 degree;
(iv) positioning the contrast pattern(s) (visual stimuli) at a predetermined distance from an eye;
(v) thereby treating and/or preventing the vision irregularity.
